(19) United States
(12) Patent Application Publication
(10) Pub. No.: US 2018/0051156 A1
(43) Pub. Date: Feb. 22, 2018

(54) POLYMER ALCOHOLYSIS

(71) Applicant: Zeus Industrial Products, Inc., Orangeburg, SC (US)

(72) Inventors: Elizabeth A. Foley, Columbia, SC (US); John Richard Campanelli, Greer, SC (US); Bruce L. Anneaux, Lexington, SC (US)

(21) Appl. No.: 15/678,272

(22) Filed: Aug. 16, 2017

Related U.S. Application Data
(60) Provisional application No. 62/375,937, filed on Aug. 17, 2016.

(51) Int. Cl. C08J 11/24; C07C 51/09; B01J 14/00; B01J 19/00; B01J 13/00; B01D 15/34

(52) U.S. Cl. CPC C08J 11/24; C07C 51/09; B01J 14/00; C08J 2367/04; B01J 13/0065; B01D 15/34; B01J 19/0066

(57) ABSTRACT

A method for enhancing/accelerating the depolymerization of polymers (e.g., those containing hydrolyzable linkages) is described herein. The disclosed method generally involves contacting a polymer comprising hydrolyzable linkages with a solvent and an alcohol to give a polymer mixture in which the polymer is substantially dissolved, wherein the contacting is conducted at a temperature at or below the boiling point of the polymer mixture. A resulting depolymerized polymer can be separated therefrom (including, e.g., monomers and/or oligomers). Such methods can be conducted under relatively mild temperature and pressure conditions.

### CROSS-REFERENCE TO RELATED APPLICATION

The present application claims priority to U.S. Provisional Patent Application No. 62/375,937, filed Aug. 17, 2016, which is incorporated herein by reference in its entirety.

### FIELD OF THE INVENTION

The present application is directed to methods for accelerating degradation of polymeric materials (e.g., polyesters such as poly(lactic acid)).

### BACKGROUND OF THE INVENTION

Poly(lactic acid) (PLA) is a thermoplastic aliphatic polyester that is ubiquitous in packaging, automotive components, medical products, and electronic components. PLA finds wide use in various fields due to properties such as high clarity, stiffness, barrier capabilities, and printability in combination with its ready processability (e.g., by extrusion, molding, film production, etc.). In addition, PLA is growing in popularity due to its “environmentally friendly” designation, as it is typically derived from plant-based materials (e.g., corn) and thus, after use, can biodegrade into its constituent organic parts. PLA is commonly derived from corn, which is fermented to give lactic acid, which is used to produce lactide, and the lactide is subjected to by ring-opening polymerization to give PLA. Due to the chiral nature of lactic acid (and lactide), various stereochemical forms of PLA can be produced. Lactic acid can be in L, D, or racemic form, and lactide can thus exist in L,L form (L-lactide), D,D form (D-lactide), or in L,D or D,L form (D,L-lactide or meso-lactide).

Due to the significant presence of PLA in such disposable end products as packaging, it is important to ensure that PLA is adequately addressed after disposal. Unlike thermoplastic resins such as polyethylene, polypropylene, polystyrene and poly(ethylene terephthalate), PLA is subject to thermal degradation and cannot be readily reprocessed (e.g., by extrusion) and thus must be separated from other plastics commonly treated in this manner. Composting PLA is possible, requiring that PLA be sent to a composting facility rather than a recycling facility, but a relatively small number of such facilities are in operation. Additionally, the supply of post-consumer PLA far exceeds the demand for compost. As such, there remains a local and worldwide need for additional methods of addressing the growing amount of post-consumer PLA.

Chemical recycling technologies, designed to recover useful chemical components from post-consumer polymer products are growing in importance. Due to the presence of ester groups along the polymer backbone, PLA can be degraded by simple hydrolysis (e.g., by reaction to break down the polymer chain into oligomers and/or monomers, which can be used for such purposes as environmentally friendly plasticizers and as feedstock for producing more PLA). However, although PLA is degradable, it biodegrades very slowly. Analysts have estimated it may take 100 to 1,000 years for a PLA bottle to decompose in a landfill under typical conditions. As such, the current state of the art in chemical recycling of PLA focuses on the use of catalysts, enzymes, and/or microbes, the application of heat and/or pressure, and/or irradiation of PLA with y-rays to promote the degradation of PLA. Such methods can be effective from a kinetic standpoint but typically involve high energy consumption and high capital costs. Accordingly, it would be beneficial to provide additional processes for enhancing the degradation of PLA.

### SUMMARY OF THE INVENTION

The present invention relates to methods for enhancing or accelerating the partial or complete degradation of polymers. In particular, the invention relates to methods for degrading polymers into smaller constituent parts (i.e., reducing the molecular weight of polymer segments to provide oligomers and/or monomers) to provide for improved recyclability of materials comprising such polymers.

In one aspect, the disclosure provides a method for depolymerizing a polymer, comprising: obtaining a polymer, wherein the polymer contains hydrolyzable linkages; obtaining a polymeric mixture by at least contacting the polymer with a solvent and an alcohol, wherein the polymer in the polymeric mixture is substantially dissolved, and wherein the contacting is conducted at a temperature at or below a boiling point of the polymeric mixture, to provide a depolymerized material. In another aspect the disclosure provides a method for converting a polymer containing hydrolyzable linkages into oligomers and/or monomers, comprising: obtaining a polymeric mixture by at least contacting the polymer with a solvent and an alcohol, wherein the polymer in the polymeric mixture is substantially dissolved, and wherein the contacting is conducted at a temperature at or below a boiling point of the polymeric mixture, to provide a depolymerized material. In such methods, the polymeric mixture can be described as exhibiting no visible phase separation to the naked eye.

The methods disclosed herein are applicable for the depolymerization of a range of different polymers. In some embodiments, the polymer comprises, at least in part, a polyester. In some embodiments, the polymer is a copolymer. In certain embodiments, the polymer is selected from a group consisting of polysaccharides; chitin; chitosan; proteins; polyglycolides; poly(caprolactones); poly(hydroxybutyrates); poly(anhydrides); aliphatic polycarbonates; polyesters, poly(orthoesters); poly(amino acids); poly(ethylene oxide); polyphosphazenes, polyvinyl alcohols, copolymers thereof, and derivatives thereof. In some embodiments, the hydrolyzable linkages within the polymer(s) subjected to the disclosed method are ester linkages. For example, specific polymers for which the disclosed methods are applicable include, but are not limited to, poly(lactic acid), derivatives of poly(lactic acid), and copolymers of poly(lactic acid). Such methods are also applicable to, e.g., poly(caprolactone), derivatives of poly(caprolactone), and copolymers of poly(caprolactone). In some embodiments, the polymer is comprised in co-mingled waste. For example, the co-mingled waste can further comprise, e.g., one or more of paper, metal, and polymers or plastics.

The solvent or solvents employed in the disclosed methods can vary and, in some embodiments, may be selected from a group consisting of methylene chloride, chloroform, tetrachloromethane, trichloroethylene, tetrahydrofuran, acetone, ethyl acetate, dioxane, N-methyl pyrrolidone, hexafluoroisopropanol, 2,2,2-trifluoroethanol, acetophenone, acetonitrile, toluene, cyclohexanone, butylbenzoate, isophorone, nitropropane, methylethyl ketone, dimethylacetamide, benzylbenzoate, caprolactone, tributyl phosphate, benzene, dimethylformamide, and any combinations thereof. The alcohol can also vary and, in some embodiments, is selected from a group consisting of C1-6 alkyl alcohols and water. One exemplary alcohol used in certain embodiments is methanol.

In certain embodiments, the polymeric mixture formed in the disclosed methods is a solution and the polymer is completely dissolved. In other embodiments, the polymeric mixture formed in the disclosed methods is a gel.

The contacting, in some embodiments, comprises substantially dissolving the polymer in the solvent in connection with obtaining a first mixture, and combining the first mixture with the alcohol in connection with obtaining the polymeric mixture. The contacting, in other embodiments, comprises combining the alcohol and the solvent in connection with obtaining a first mixture, and combining the first mixture with the polymer in connection with obtaining the polymeric mixture.

In some embodiments, the contact between the polymer, solvent, and alcohol occurs without a depolymerization catalyst. Optionally, the disclosed methods may, in some embodiments, employ a depolymerization catalyst. As such, in some embodiments, the contacting step of the disclosed methods can further comprise adding a depolymerization catalyst. Exemplary depolymerization catalysts include, but are not limited to, metal oxides and organometallic catalysts.

The temperature at which the contacting is conducted, in some embodiments, is about 1° C. to about 20° C. below the boiling point of the polymeric mixture, about 5° C. to about 25° C. below the boiling point of the polymeric mixture, or about 10° C. to about 25° C. below the boiling point of the polymeric mixture. In some embodiments, the contacting occurs at about atmospheric pressure.

In some embodiments, the disclosed method further comprises obtaining the depolymerized material. Methods for obtaining the depolymerized material include, in some embodiments, one or more of distillation, extraction, precipitation, recrystallization, and chromatography. In some embodiments, the method further comprises recovering the solvent and/or alcohol for reuse.

In particular embodiments, the polymer comprises poly(lactic acid) and the depolymerized material comprises one or more lactate monomers, one or more lactate oligomers, or any combination thereof. In particular embodiments, the polymer comprises poly(lactic acid) and the depolymerized material comprises one or more lactic acid monomers, one or more lactic acid oligomers, or any combination thereof. In certain embodiments, a molecular weight distribution of the depolymerized material is less than a molecular weight distribution of the polymer.

The present disclosure also provides a depolymerized polymer prepared according to the methods disclosed herein. In some embodiments, the depolymerized material, oligomers, and/or polymers can be readily separated from the solvent mixture. Exemplary methods for such separation include, but are not limited to one or more of distillation, extraction, precipitation, recrystallization, and chromatography. The depolymerized material, typically comprising oligomers, and/or monomers, can be further used, e.g., as environmentally-friendly plasticizers or as feedstock for re-polymerizing monomers into polymers.

The disclosure further provides, in another aspect, a system comprising: one or more mixing modules configured to obtain the polymer comprising hydrolyzable linkages, and to obtain a polymeric mixture by at least contacting the polymer with a solvent and an alcohol; and one or more reactors configured to provide a depolymerized material based at least in part by conducting contacting between the polymer, the solvent and the alcohol at a temperature at or below a boiling point of the polymeric mixture such that the polymer is substantially dissolved.

The system can comprise various features. For example, in some embodiments, at least one of the one or more mixing modules obtain the polymer based at least in part on receiving the polymer from an input source. In some embodiments, at least one of the one or more mixing modules obtain the polymer based at least in part on retrieving the polymer from an input source. In some embodiments, at least one of the one or more reactors comprises at least one of the one or more mixing modules. In some embodiments, at least one of the one or more reactors is a continuous stirred tank reactor, a batch reactor, or a plug flow reactor.

Various additional features can be incorporated within the disclosed system. For example, in some embodiments, the system further comprises one or more heating modules configured to provide heat to the polymeric mixture. In some embodiments, at least one of the one or more reactors comprises, or is operatively connected to, at least one of the one or more heating modules. The system can, in some embodiments, further comprise one or more separating modules configured to separate the depolymerized material. In some embodiments, the depolymerized material is separated from one or more of an amount of solvent and an amount of alcohol. In some embodiments, one or more of at least a part of the amount of solvent and at least a part of the amount of alcohol is provided to at least one of the one or more reactors. In some embodiments, one or more of at least a part of the amount of solvent and at least a part of the amount of alcohol is provided to at least one of the one or more mixing modules. The one or more separating modules, in some embodiments, separate the depolymerized material based at least in part on one or more characteristics of one or more of the depolymerized material, the solvent, and the alcohol. The one or more separating modules, in some embodiments, separate the depolymerized material based at least in part on one or more characteristics of at least one of one or more components to be separated. In some embodiments, at least one of the one or more separation modules comprises a distillation column. In some embodiments, the one or more separating modules is operatively connected to the one or more reactors. The system, in some embodiments, further comprises a size reducing module configured to reduce a size of a material from which the polymer is obtained. The system, in some embodiments, further comprises a washing module configured to clean a material from which the polymer is obtained.

The disclosure further provides a depolymerized material obtained by contacting a polymer comprising hydrolyzable linkages, a solvent, and an alcohol, wherein the contacting is conducted at a temperature at or below a boiling point of a polymeric mixture comprising the polymer, the solvent, and the alcohol, and wherein the polymer in the polymeric mixture is substantially dissolved. In some embodiments, the depolymerized material is one or more of lactic acid, methyl lactate, poly(lactic acid) oligomers, and lactate oligomer.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise. Other aspects and advantages of the present invention will become apparent from the following.

### BRIEF DESCRIPTION OF THE DRAWINGS

In order to provide an understanding of embodiments of the invention, reference is made to the appended drawings, which are not necessarily drawn to scale, and in which reference numerals refer to components of exemplary embodiments of the invention. The drawings are exemplary only, and should not be construed as limiting the invention.

FIG. 1 provides a schematic representation of one embodiment of the method disclosed herein;

FIG. 2 provides a schematic representation of a second embodiment of the method disclosed herein; and

FIG. 3 provides a schematic representation of an embodiment of a system according to the present disclosure.

### DETAILED DESCRIPTION OF THE INVENTION

The present invention will now be described more fully hereinafter with reference to the accompanying figures, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

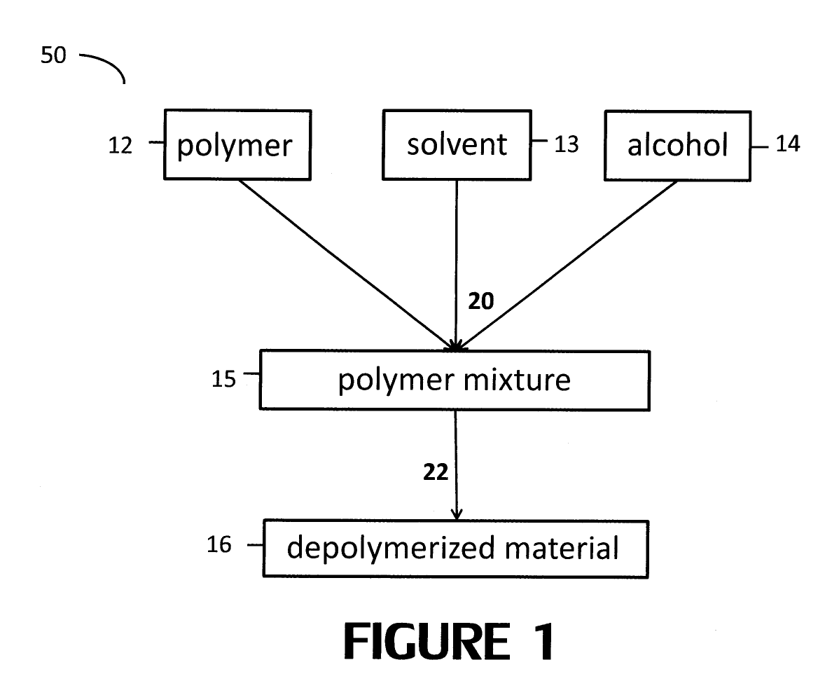

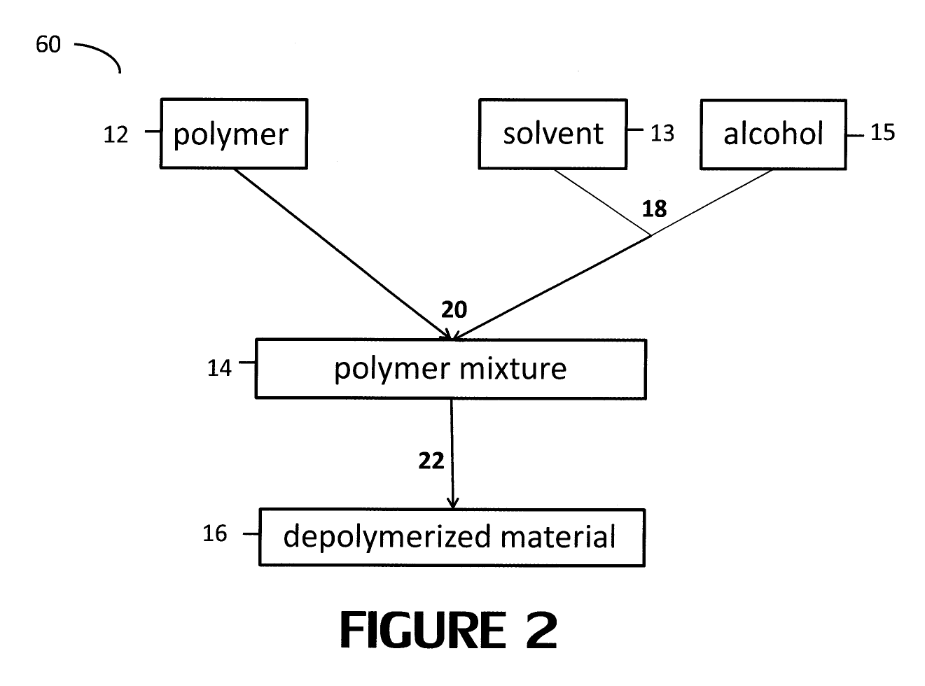

The disclosure relates generally to methods for degrading or reducing the molecular weight of various polymers, including polyesters such as poly(lactic acid) (PLA), derivatives thereof, and copolymers thereof. The disclosed methods can be conducted at relatively low temperatures, ambient pressures, and can be conducted in the absence of added catalysts (although, in some embodiments, addition of a catalyst may be beneficial to further enhance the degradation). Exemplary, schematic overview of the degradation processes 50 and 60 outlined herein are provided in FIGS. 1 and 2. The disclosed methods generally employ conditions that uniquely allow an alcohol component to access hydrolyzable linkages within the polymer chain, leading to chain scission reactions under relatively mild temperature and pressure. As such, at least partial dissolution of the polymer and/or at least partial miscibility of the alcohol to facilitate such access is important to obtain the results detailed herein; however, the methods to achieve the requisite access can vary, as detailed further herein below.

The disclosed methods generally provide the polymer 12 in a form sufficient for the alcohol 14 to access hydrolyzable linkages (e.g., ester linkages) present therein. According to the present disclosure, the degradation of polymer 12 can be enhanced generally by substantially dissolving (e.g., dissolving) the polymer in a reaction mixture comprising an alcohol 14, as will be described in further detail herein below. Typically, the alcohol 14 is substantially miscible (e.g., miscible) in the reaction mixture. The “substantial dissolution” (e.g., dissolution) of the polymer 12 and the “substantial miscibility” of the alcohol 14 can be achieved at any stage of the disclosed process, as will be more fully detailed herein. Surprisingly, in all such methods, the addition/inclusion of the alcohol effectively reduces the molecular weight of the polymer (i.e., degrades the polymer chain into smaller polymer chains and/or oligomers and/or monomer units) under relatively mild conditions.

The polymer 12 subjected to the disclosed processes can vary. It is noted that, although the present application focuses on the degradation of PLA, the methods described herein are applicable to other polymers. The methods disclosed herein may be applicable particularly to PLA and other aliphatic polyesters. In some embodiments, such methods are applicable to hydrolyzable polymers including, but not limited to, polysaccharides (e.g., dextran and cellulose); chitin; chitosan; proteins; polyglycolides; poly(caprolactones); poly(hydroxybutyrates); poly(anhydrides); aliphatic polycarbonates (e.g., polytrimethyl carbonate); poly(orthoesters); poly(amino acids); poly(ethylene oxide); polyphosphazenes, and polyvinyl alcohols. The methods can also be applicable to copolymers comprising two or more of the types of polymers described herein, in block, random, or alternating form. “Copolymer” as used herein is not intended to be limited to two types of polymers and can include any number of different monomer units (e.g., terpolymers, tetrapolymers, and the like). Further, the methods can apply to copolymers comprising at least some hydrolyzable polymer units and to derivatives and/or blends of any of the degradable polymers referenced herein. The method is not understood to be limited in terms of the molecular weight of the polymer to be degraded. As such, polymers of varying molecular weights and varying polydispersity indices can be degraded in accordance with the methods provided herein.

The polymer 12 subjected to the methods of the present disclosure can be in various forms. For example, in some embodiments, the methods disclosed herein are applicable in the context of breaking down disposable products comprising polymers after use (e.g., polymer-based packaging, cups, plates, utensils, and the like). Polymer 12 can thus be present within waste products comprising one or more of the polymers generally disclosed herein above. Polymer 12 can also be present within a waste stream that has already been processed in some manner, which comprises one or more of the polymers generally disclosed herein above. Accordingly, the material subjected to the methods outlined herein may comprise substantially pure polymer or can comprise one or more components in addition to polymer 12. As such, it is generally understood that the terms used herein, unless otherwise indicated, refer specifically to polymer 12 and not, e.g., to other components present within the sample comprising polymer 12, which sample is subjected to the methods disclosed herein. For example, “substantial dissolution” of polymer 12 implies only that the polymer 12 is substantially dissolved; however, the resulting material may not be a solution, as other components in the sample subjected to the method (e.g., where polymer 12 is provided in the form of a waste stream) may not be substantially dissolved, e.g., within polymer mixture 15.

In disclosed methods 50 and 60, the polymer 12 is combined with one or more solvents 13 and one or more alcohols 15 in step 20 to provide a polymer mixture 15. The order of addition of these three components can vary. FIG. 1 shows one exemplary embodiment, i.e., Method 50, wherein the polymer, solvent, and alcohol are independently added to form the reaction mixture. However, it is to be understood that these components can be added in any order and in any combination to obtain polymer mixture 15. FIG. 2 shows one exemplary embodiment, i.e., Method 60, wherein the solvent and alcohol are first combined in step 18 and then this mixture is combined with polymer 12 to form polymer mixture 15.

The polymer mixture 15 can be in various forms. In some embodiments, the polymer 12 is in substantially dissolved form within polymer mixture 15. “Substantially dissolved” as used herein means that the little to no undissolved/particulate polymer material 12 is present in the polymer mixture 15. In particular, a “substantially dissolved” mixture will be considered to exhibit no visible phase separation to the naked eye. A “substantially dissolved” mixture can also be characterized by exhibiting no discrete layers and no interface between separate components to the naked eye. The polymer mixture may be in any form exhibiting these characteristics. Although not limited thereto, in such embodiments, polymer mixture 15 can be a solution. In some embodiments, polymer mixture 15 can be a gel, which intended herein to have its typical meaning in the art. “Substantially dissolved” as used herein is intended to encompass solutions, gels, and other forms that satisfy the definitions referenced above.

It is noted that, in some embodiments, particularly where the material subjected to disclosed process 50 or 60 (comprising polymer 12) includes components other than the polymer to be processed according to the disclosed method, such components other than the polymer to be processed may, in some embodiments, be largely undissolved, resulting in a dispersion/suspension of those other components. Combining step 20 may in some embodiments comprise agitation and/or heating to promote substantial dissolution of the polymer 12 in polymer mixture 15. The present invention, in some embodiments, is particularly suited to removing PLA-based materials from recycle streams containing multilayer packaging or co-mingled plastic waste. Co-mingled waste is understood in the conventional sense of relating to mixtures of different waste materials. The polymer 12 (e.g., PLA) is preferentially dissolved when subjected to the disclosed process, whereas the remaining largely insoluble waste plastics can be removed from the solution by flotation, sedimentation, filtration or other means commonly employed in the recycling industry. The largely insoluble waste plastics thus separated from the polymer 12 (e.g., PLA)-based materials can be subjected to further processing by a variety of methods known in the art

In some embodiments, the polymer 12 is not in a substantially dissolved form within polymer mixture 15 and, in such embodiments, polymer mixture 15 can be, e.g., a suspension/dispersion. Again, substantial dissolution of polymer 12 is central to the disclosed process; however, where this is not achieved upon mixing the components, it may be achieved during reaction (i.e., during step 22).

The one or more solvents 13 can vary and may, in some embodiments, be any one or more solvents capable of substantially dissolving the polymer 12 at room temperature and/or under reaction conditions employed in step 22. The specific solvent or solvents chosen to substantially dissolve the polymer can vary and depends on the specific polymer or polymers 12 to be degraded. In some embodiments, the solvent is an “inert” solvent, meaning that it does not actively participate in alcoholysis/hydrolysis of the polymer 12. However, the invention is not limited thereto and, in some embodiments, the solvent 13 can actively participate to at least some extent in alcoholysis/hydrolysis of the polymer (e.g., where the solvent comprises one or more —OH groups, in which case it may function as both the “solvent” component and the “alcohol component”).

Where the polymer is PLA or a copolymer or derivative thereof, one of skill in the art will appreciate that common solvents known to dissolve PLA include, but are not limited to, methylene chloride, chloroform, tetrachloromethane, trichloroethylene, tetrahydrofuran (THF), acetone, ethyl acetate (EtOAc), dioxane (e.g., 1,4-dioxane), N-methyl pyrrolidone (NMP), hexafluoroisopropanol, 2,2,2-trifluoroethanol, acetophenone, acetonitrile, toluene, cyclohexanone, butylbenzoate, isophorone, nitropropane, methylethyl ketone (MEK), dimethylacetamide, benzylbenzoate, caprolactone, tributyl phosphate, benzene, dimethylformamide (DMF), and mixtures thereof and, accordingly, such solvents are understood to be useful for this purpose. The concentration of the polymer(s) 12 in polymer mixture 15 can vary and is not particularly limited, other than that the concentration limit posed by the solubility limit of the polymer in the particular solvent (at room temperature and/or at the temperature employed in step 22).

The alcohol 14 of methods 50 and 60 is generally understood to be any alcohol capable of effectuating chain scission of at least a portion of the linkages present within the polymer 12. An alcohol is generally any reagent having a free hydroxyl (—OH) group. It is believed that, according to the methods outlined herein, degradation/depolymerization is effected by alcoholysis, i.e., chain scission by the alcohol (e.g., “hydrolysis” where the alcohol is water, and “methanolysis” where the alcohol is methanol). The specific alcohol 14 can be, for example, water or an alkyl alcohol, including but not limited to, C1-6 alkyl alcohols, such as methanol (MeOH), ethanol (EtOH), 1-propanol, 2-propanol (isopropanol), 1-butanol (n-butyl alcohol), 2-butanol (sec-butyl alcohol), 3-methyl-1-propanol (isobutyl alcohol), 2-methyl-2-propanol (t-butyl alcohol), pentanols, and hexanols. The alcohol in some embodiments may be a glycol or a hydroxide salt (e.g., including, but not limited to, NaOH or KOH).

As disclosed herein above, the alcohol 14 is advantageously substantially miscible in the reaction mixture (and is thus typically miscible in solvent 13 and/or is miscible in a mixture of polymer 12 and solvent 13) in at least one stage of the disclosed process. Accordingly, in some embodiments, polymer mixture 15 comprises a single liquid phase, i.e., the alcohol is substantially miscible in polymer mixture 15. In some such embodiments, the alcohol co-solvent 14 may be used to help solubilize the polymer 12 such that a broader range of solvents 13 can be used to provide polymer 12 in a substantially dissolved form within polymer mixture 15. In other embodiments, alcohol 14 is not readily miscible with the other components of polymer mixture 15 (giving, e.g., a biphasic polymer mixture 15) and the substantial miscibility is achieved under the conditions of reaction step 22.

The conditions at which these three (or more) components (comprising polymer 12, solvent 13, and alcohol 14) are reacted in step 22 can vary. The ratio of polymer: solvent: alcohol can vary, so long as sufficient solvent and/or alcohol is provided to ensure that the polymer 12 is substantially dissolved during at least a portion of step 22 and that alcohol 14 is substantially miscible during at least a portion of step 22. Typically, the volume of solvent 13 is greater than the volume of alcohol to ensure that the polymer is substantially dissolved; however, this is not required. In certain embodiments, the amounts of polymer, solvent, and alcohol are maintained as a constant throughout the reaction step 22, but in other embodiments, addition of more alcohol 14 during reaction step 22 may be advantageous to further enhance the depolymerization of polymer 12.

Typically, the alcohol 14 remains in contact with polymer 12 and solvent 13 during reaction step 22 at least for a period of time sufficient to degrade at least a portion of the polymer 12 (i.e., to reduce the molecular weight of at least a portion of the polymer). The time required to achieve the desired level of depolymerization can vary, as will be recognized by one of skill in the art, e.g., depending upon the specific reaction conditions employed (i.e., parameters such as temperature and pressure can be modified to increase/decrease the rate of reaction as desired).

The temperature at which these components are contacted is advantageously rather low, i.e., below the boiling point of polymer mixture 15 (i.e., the mixture comprising polymer 12, solvent 13, and alcohol 14). Although the reactions disclosed herein can be effective at room temperature, such reactions are generally conducted at a temperature that is elevated with respect to room temperature to increase reaction rate. As such, heat is advantageously applied during step 22 to the mixture of polymer, solvent, and alcohol to raise the temperature above room temperature to a temperature at or below the noted boiling point of the polymer mixture in its entirety (in some embodiments, up to the boiling point, in some embodiments, up to about 2° below the boiling point, in some embodiments up to about 5° below the boiling point, in some embodiments up to about 10° below the boiling point of the mixture). One of skill in the art is generally aware that the boiling point of a mixture of components can differ from the boiling point of any one component and can readily calculate or determine experimentally the boiling point of any given mixture to ensure that the method disclosed herein is conducted at the temperatures described herein above. One of skill in the art further recognizes that, where the mixture of components has a high concentration of one component, the boiling point of the mixture can be close to the boiling point of that component. As one example, where an excess of chloroform is employed as solvent 13, the heat applied during step 22 may be sufficient to raise the temperature of the mixture, e.g., to less than about 60° C., less than about 58° C., or less than about 55° C., e.g., about 40° C. to about 60° C. or about 50° C. to about 60° C. In some embodiments, the temperature of the mixture is sufficient to ensure at least substantial dissolution of polymer 12 during the reaction.

The pressure at which step 22 is conducted can also vary but according to the methods disclosed herein, the pressure is preferably at or near atmospheric pressure. In some embodiments, the pressure is considered low to moderate, e.g., less than or equal to about 10 bar, less than or equal to about 6 bar, or less than or equal to about 4 bar. One of skill in the art understands that pressure and temperature are directly related. For example, where the mixture boils at or near the boiling point of chloroform, the temperature required for boiling at 1 bar pressure is about 61° C., the temperature required for boiling at 10 bars pressure is about 120° C., and the temperature required for boiling at 0.1 bar pressure is about 18° C. As such, selection of appropriate reaction conditions for step 22 requires consideration of mixture components, pressure, and temperature to achieve a substantially dissolved polymeric material maintained below the boiling point of the mixture. Advantageously, the methods disclosed herein generally do not require high pressure vessels for reaction.

In some embodiments, no depolymerization catalyst is required in step 22 and this step is conducted in the absence of added depolymerization catalysts. Advantageously, the only three components in contact during reaction step 22 in such embodiments are the polymer 12, solvent 13, and alcohol 14. It is noted that, in some embodiments, residual catalyst from polymer production may be present in the mixture (as polymer 12 may comprise such residual catalyst); however, advantageously, no additional catalyst is necessarily added to effectuate the depolymerization acceleration described herein. However in some embodiments, the methods outlined herein can be used in combination with a depolymerization catalyst, e.g., to further enhance or speed up the depolymerization process. Various depolymerization catalysts are known, e.g., as disclosed in International Patent Application Publication No. WO 2015/112098 to PTT Public Company Ltd., which is incorporated herein by reference in its entirety. Such catalysts include, but are not limited to, metal oxides, e.g., antimony trioxide, or organometallic compounds having 1-20 carbon atoms, such as organometallic tin or titanium compounds.

The exact composition of the depolymerized material 16 produced by the described methods 50 and 60 can vary. The exact composition of the monomers and/or oligomers produced from a given mixture can vary, depending in particular on the composition of polymer 12 and/or on the particular alcohol 14 employed in the reaction. As an example, where polymer 12 comprises PLA, the depolymerized material 16 generally comprises lactate monomer and oligomers. The depolymerized material 16 can, in some embodiments, comprise at least about 50% by dry weight monomers and oligomers, at least about 75% by dry weight monomers and oligomers, at least about 90% by dry weight monomers and oligomers, at least about 95% by dry weight monomers and oligomers, or at least about 99% by dry weight monomers and oligomers. Again, these values do not take into account other components that may have been present within the sample subjected to process 50 or 60 which comprises polymer 12 (e.g., where polymer 12 is provided within a waste stream).

In some embodiments, the polydispersity index (PDI, a measure of the molecular weight distribution, calculated based on M_{w}/Mₙ) of the depolymerized material 16 is lower than the original PDI of the polymer 12. When the depolymerization reaction has gone to completion (i.e., all hydrolyzable linkages have been cleaved) or largely to completion, a narrow molecular weight distribution would be expected (e.g., close to 1, indicating most components with the same molecular weight, e.g., oligomers and/or monomers). In some embodiments, the PDI of the depolymerized material is roughly comparable to the original PDI of the polymer 12 (e.g., plus or minus about 50% of the polymer PDI, plus or minus about 25% the polymer PDI value, or plus or minus about 10% of the polymer PDI value). In some embodiments, the polymer 12 subjected to the disclosed method may not exhibit a discrete sample, i.e., it may comprise a range of molecular weights, including a range of molecular weights exhibiting a bimodal (or further multimodal) molecular weight distribution. In such embodiments, one would expect to see a similar bimodal (or multimodal) distribution during the reaction 22 and which may be bimodal or unimodal following reaction.

In certain embodiments, the production of oligomers is advantageous, as certain such compounds have industrial significance as plasticizers or components of pharmaceutical coatings and formulations. The production of monomers is also advantageous as such monomers can be recovered and used, e.g., as feed stock for the production of more PLA. Products (i.e., monomers and/or oligomers) can generally be easily obtained from depolymerized material 16, by methods including but not limited to, distillation or evaporation of residual solvent and alcohol from depolymerized material 16, extraction of the products from depolymerized material 16, or by precipitation methods, followed, e.g., by filtration or centrifugation to separate solid product from solvent and alcohol. In certain embodiments, monomer (e.g., lactide monomer from PLA degradation) and/or oligomers can be further purified, e.g., by recrystallization using known methods prior to reuse. The methods outlined herein are beneficial due to the fact that, as in some embodiments, no depolymerization catalyst is intentionally added to the reaction mixture, one less component is present in the final product to be separated from the depolymerized polymer to provide the purified depolymerized material, thus simplifying the purification.

In addition, residual solvent and/or alcohol can typically be recovered from such processes. For example, where distillation is used to isolate depolymerized material 16, the distillate can be condensed to provide a mixture of solvent and alcohol and these can be further separated, e.g., by distillation. As such, the present disclosure in some embodiments provides a closed, environmentally friendly process via recovery of residual solvent and/or reactant. In certain embodiments, recovered solvent 13 and/or alcohol 14 can be reused in processes 50 and 60 to depolymerize additional polymer 12.

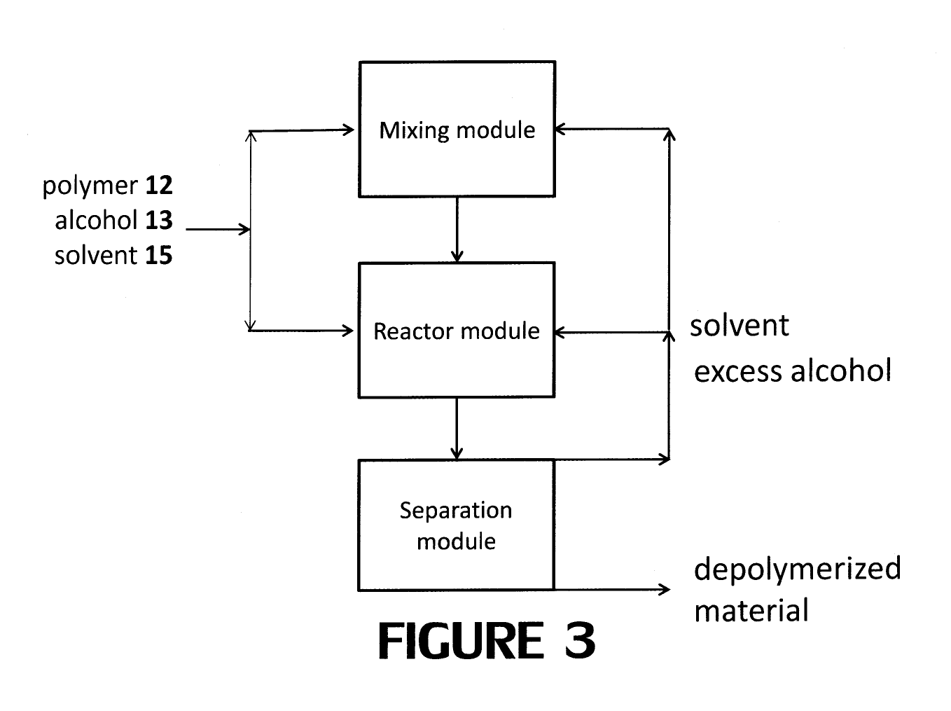

A person of skill in the art could readily conceive of a number of processes to carry out the various embodiments of this invention and to various systems for conducting the disclosed methods and processes. One suitable system is shown in FIG. 3, which shows the implementation of a mixing module, reactor module, and separation module to accomplish the various steps of the methods disclosed herein and provide a depolymerized material.

One such process could comprise a mixing tank wherein a co-mingled stream of plastics is contacted with a suitable solvent to substantially solubilize polymer 12, e.g., PLA, while the remaining materials in the co-mingled stream remain unsolubilized and are removed by flotation, sedimentation, or filtration. The substantially solubilized polymer 12 (e.g., PLA) and solvent could further be transported into a batch or continuous stirred tank reactor, where the alcohol is added along with heat and catalyst to carry out the depolymerization reaction. The resulting oligomers and monomers (e.g., PLA oligomers and methyl lactate monomers) could further be transported to a separation module such as a distillation column wherein the depolymerized product would be separated from the excess alcohol and solvent. It can further be envisaged that the excess alcohol and solvent would be recirculated to one or both of the mixing module or reactor module to make an economically and environmentally advantageous process.

The methods disclosed herein can also be conducted on a smaller, e.g., laboratory scale. For example, in one embodiment, the depolymerization system contains a container such as a round bottom flask containing the reaction components connected to a distillation column. The distillation column is also connected to a receiving flask, e.g., with a stopcock separating the column from the receiving flask. The container (e.g., round bottom flask) is heated, e.g., by partially or fully submerging it into a heated oil bath. In some embodiments, a magnetic stir bar can be added to the container (e.g., round bottom flask) and the container (e.g., round bottom flask) can be placed on a stir plate.

The disclosed method for depolymerization of polymers provided herein leads to unexpected results with respect to common understanding in the art of polymer chemistry. For example, one of skill in the art is generally aware that alcohols are commonly used for precipitation of polymers (e.g., including, but not limited to, polyesters). See, for example, S. Lee et al., J. Polym. Sci.: Part A: Polymer Chemistry Vol. 39, pp. 973-985 (2001) (describing, on page 975, precipitation of polylactide by pouring a chloroform solution thereof into an excess of methanol), and S. Kaihara et al., Nature Protocols Vol. 2, No. 11, pp. 2767-2771 (2008) (describing, on page 2769, dissolution of a PLLA reaction product in chloroform and precipitation of the polymer by adding the solution into an excess amount of methanol), which are both incorporated herein by reference.

### Experimental

Molecular weights as reported herein were evaluated using gel permeation chromatography (GPC)/size exclusion chromatography (SEC) with refractive index (RI) detection in chloroform. Molecular weights were determined from the RI chromatograms for each sample by comparison with a RI chromatogram calibration curve based on polystyrene standards of known molecular weights ranging from 162 to 6,035,000 g/mol. Inherent viscosity (IV) values reported herein were measured using: chloroform at 25° C., the ASTM D2857 procedure “Standard Practice for Dilute Solution Viscosity of Polymers”, and a YSI Incorporated AVS 370 semi-automated viscosity measurement system consisting of AVS 370 measuring unit, AVS/SK optical measuring stand, CT 72 thermostatic water bath, a Ubbelohde 0c viscometer and Win Visco 370 software.

### EXAMPLE 1

Solution A, consisting of PLA, methanol (MeOH), and chloroform (CHCl₃) (4/25/71 w/w/w) was prepared and left at 57° C. overnight. After this time, it was noted that the solution exhibited lower viscosity than previously, and the molecular weight of the sample was evaluated.

Two additional, similar solutions were prepared using the same PLA but with PLA/methanol/chloroform ratios of 11/25/64 w/w/w (Solution B) and 7.5/25/67.5 w/w/w (Solution C). Solutions A, B, and C were maintained at 57° C. for 7 days, air-dried to evaporate residual methanol and chloroform, and then further dried in a vacuum oven for 7 days. The inherent viscosities of these solutions were measured and compared with a control (“Control”) prepared by evaporating a 10% solution (w/w) of the same PLA in chloroform only. The results are provided below in Table 1.

TABLE 1
Data for Depolymerized Products

| | Sample (PLA/MeOH/CHCl₃) wt./wt./wt. | | | |
|---|---|---|---|---|
| | 10/0/90 | 4/25/71 | 7.5/25/67.5 | 11/25/64 |
| | (Control) | (Solution A) | (Solution B) | (Solution C) |
| Mean IV (dL/g) | 4.0 | 1.1 | 0.6 | 1.6 |

All samples showed significant reduction in IV, consistent with a large drop in the molecular weight of the PLA solute in the presence of methanol, believed to cause chain scissions in the PLA.

### EXAMPLE 2

To study the kinetics of the degradation (methanolysis) reaction, a ternary solution was made up in a glass jar at room temperature at the following loading: PL49/MeOH/CHCl₃, 11.1/26.0/62.9 (w/w/w). PL49 is a commercially available PLLA from Corbion Purac (Amsterdam, the Netherlands), sold under the tradename PURASORB® PL49 (inherent viscosity=4.9 dL/g).

The mixture was a gel at room temperature and was placed on a shaker table at 100 RPM to homogenize for 66 hours. The kinetic study was begun when a portion of the ternary mixture was placed in an incubator set at 57° C. The opaque gel turned into a clear solution after several minutes at this temperature. Samples were withdrawn at various time points as indicated in the table below. Samples were prepared as described previously and submitted for IV testing.

TABLE 2
Data on Kinetics of Depolymerization Analysis

| | Description | Time at | Time at | |
|---|---|---|---|---|
| Sample | (PL49/MeOH/CHCl₃) | 23° C. | 57° C. | Mean IV, |
| ID | wt./wt./wt. | (hours) | (hours) | dL/g |
| PLA-1 | 11.1/26.0/62.9 | 66 | 0 | 2.6 |
| PLA-2 | 11.1/26.0/62.9 | 66 | 2 | 0.67 |
| PLA-3 | 11.1/26.0/62.9 | 66 | 8 | 0.2 |
| PLA-4 | 11.1/26.0/62.9 | 66 | 75 | 0.1 |
| PLA-5 | 11.1/26.0/62.9 | 141 | 0 | 2.8 |
| (control) | | | | |

Sample PLA-1 provides the Mean IV for a homogenized material (time=0 of the kinetic study) and was used as a reference. For the control sample (132-5) maintained at room temperature for 75 hours beyond the 66 hours homogenization period, there is essentially no change in IV after this time (2.8 versus 2.6 dL/g). Each of samples PLA-2, PLA-3, and PLA-4 was heated at 57° C. for varying amounts of time, and the results of Table 2 show that significant reductions in PLA molecular weight occurred very quickly at 57° C. for all heated samples. This drastic reduction in IV at 57° C. (from 2.6 to 0.67 after 2 hours) is indicative of rapid chain scission. The presented IV data is intended to demonstrate a clear general trend toward decreasing IV of mixtures comprising polymer (here, PLA), solvent (here, CHCl₃), and alcohol (here, MeOH) at the indicated conditions. This table is thus not to be relied on as providing absolute values; however, it is clearly reflective of the overall trend, as further demonstrated in Example 3, below.

### EXAMPLE 3

In order to evaluate the change in molecular weight distribution (MWD) resulting from the depolymerization methods, selected samples were sent out for gel permeation chromatography (GPC), as outlined below in Table 3. The samples were prepared and air-dried for 21 days before evaluation, and not vacuum-dried, which can pull off the lactate monomer. Results from the GPC analysis are tabulated below in Table 4.

TABLE 3
Samples Studied in Depolymerization MWD Analysis

| Sample ID | Description (PL49/MeOH/CHCl₃) |
|---|---|
| PLA-6 | PL49 polymer control, cast from a 10% |
| | solution with CHCl₃ |
| PLA-7 | Cast from solution PLA-4 (see Table 2, |
| | i.e., 75 h at 57° C.) |
| PLA-8 | Cast from solution PLA-5 (see Table 2, |
| | i.e., 75 h at room temperature) |
| PLA-9 | Methyl lactate monomer control |
| PLA-10 | PL49 polymer control (resin pellets) |

TABLE 4
GPC studies of Depolymerization Products

| | | Molar Mass Average (g/mol) | | | M_{w}/Mₙ |
|---|---|---|---|---|---|
| Sample ID | Injection | Mₙ | M_{w} | M_{z} | (PDI) |
| PLA-6 | 1 | 179,400 | 413,200 | 866,500 | 2.30 |
| | 2 | 170,400 | 417,300 | 942,400 | 2.45 |
| | Average | 174,900 | 415,250 | 904,450 | 2.38 |
| | Std. Dev. | 6,364 | 2,899 | 53,669 | 0.10 |
| PLA-7 | 1 | 1,737 | 3,509 | 6,149 | 2.02 |
| | 2 | 1,814 | 3,598 | 6,278 | 1.98 |
| | Average | 1,776 | 3,554 | 6,214 | 2.00 |
| | Std. Dev. | 54 | 63 | 91 | 0.03 |
| PLA-8 | 1 | 137,000 | 333,200 | 699,400 | 2.43 |
| | 2 | 125,700 | 320,400 | 727,200 | 2.55 |
| | Average | 131,350 | 326,800 | 713,300 | 2.49 |
| | Std. Dev. | 7,990 | 9,051 | 19,658 | 0.08 |
| PLA-10 | 1 | 188,100 | 529,300 | 1,341,000 | 2.81 |
| (control) | 2 | 185,500 | 503,000 | 1,224,000 | 2.71 |
| | Average | 186,800 | 516,150 | 1,282,500 | 2.76 |
| | Std. Dev. | 1,838 | 18,597 | 82,731 | 0.07 |

Table 4 summarizes the various parameters related to polymer molecular weight distribution for the samples listed in Table 3: the number average molecular weight, Mₙ, the weight average molecular weight, M_{w}, and the z-average molecular weight, M_{z}. It also gives the polydispersity index, PDI, or the ratio M_{w}/Mₙ, which is an indication of the breadth of the molecular weight distribution. From Table 4, it can be noted that there is about a 6.5% difference in Mₙ between the resin pellets (PLA-10) and the cast control film (PLA-6). The values of PDI for the two materials are within 15%. Hence, there has been little degradation in polymer molecular weight due to the process of solvating the resin pellets into chloroform. The sample cast from the ternary solution (containing methanol) that was held at room temperature (PLA-8) shows about a 25% drop in Mₙ relative to the cast control (PLA-6), indicating that some degradation has occurred even at room temperature. However, the sample cast from the ternary solution (containing methanol) that was held at 57° C. (PLA-7) shows a dramatic 98% reduction in Mₙ, indicating that significant degradation has occurred at the elevated temperature. The observation that PDI varies only from 2.38 to 2.00 for these samples is consistent with random chain scission being the prevalent depolymerization mechanism.

### EXAMPLE 4

Various studies were conducted to determine the effect, if any, of various reaction parameters, as outlined generally below in Table 5 and as described in the following paragraph. For these studies, a number of resins was used including NW4042, a packaging-grade PLA resin manufactured by Natureworks LLC., and Capa 6800, a poly(caprolactone) resin from Perstorp.

TABLE 5
Data on Kinetics of Further Depolymerization Analysis

| | | Time at | Time at | Mean | | |
|---|---|---|---|---|---|---|
| | | 23° C. | 57° C. | IV, | Mₙ | M_{w}/Mₙ |
| Sample ID | Description | (hours) | (hours) | dL/g | (g/mol) | (PDI) |
| PLA-11 | 11.1/26.0/62.9 | 48 | 75 | 0.8 | 38,800 | 2.39 |
| | PL49/MeOH/CHCl₃ | | | | | |
| | wt./wt./wt. in 250 mL reactor | | | | | |
| PLA-7 | 11.1/26.0/62.9 | 66 | 75 | 0.1 | 1,776 | 2.00 |
| | PL49/MeOH/CHCl₃ | | | | | |
| | wt./wt./wt. in 10 mL reactor | | | | | |
| PLA-12 | 11.1/26.0/62.9 | 48 | 150 | 0.3 | 12,200 | 2.37 |
| | PL49/MeOH/CHCl₃ | | | | | |
| | wt./wt./wt. in 250 mL reactor | | | | | |
| PLA-13 | 11.1/26.0/61.9/1 | 48 | 7 | 0 | N/A | N/A |
| | PL49/MeOH/CHCl₃/Sn(II)Oct₂ | | | | | |
| | wt./wt./wt./wt. | | | | | |
| PLA-14a | Lower IV PLA resin (NW4042) | — | — | 1.3 | 43,100 | 3.18 |
| (Control) | Pellets alone | | | | | |
| PLA-14b | 11.1/26.0/62.9 | | | | | |
| | NW4042/MeOH/CHCl₃ | 48 | 75 | 0.9 | ND | ND |
| | wt./wt./wt. | | | | | |
| PLA-15 | 11.1/26.0/62.9 | 0 | 402 | 0.6 | 8,700 | 3.82 |
| | NW4042/MeOH/THF | | | | | |
| | wt./wt./wt. | | | | | |
| PLA-16 | 11.1/26.0/62.9 | 0 | 402 | 0.4 | 5,700 | 4.38 |
| | NW4042/water/THF | | | | | |
| | wt./wt./wt. | | | | | |
| PLA-17 | 11.1/26.0/62.9 | 0 | 75 | 0.8 | 22,300 | 3.70 |
| | NW4042/water/THF | | | | | |
| | wt./wt./wt. | | | | | |
| PLA-18 | 11.1/26.0/62.9 | 48 | 75 | 1.8 | 51,650 | 4.15 |
| | PL49&NW4042/MeOH/CHCl₃ | | | | | |
| | wt./wt./wt. | | | | | |
| | (PL49:NW4042 = 1:1) | | | | | |
| PET-1a | Bottle flakes alone | — | — | — | 37,300 | 2.2 |
| PET-1b | Bottle flakes recovered from | — | 75 | — | 37,800 | 2.2 |
| | PLA/CHCl₃/MeOH mixture | | | | | |
| PCL- | Capa6800 PCL resin pellets | — | — | 2.5 | ND | ND |
| Control | | | | | | |
| PCL-1 | 11.1/26.0/62.9 | 48 | 75 | 2.2 | ND | ND |
| | Capa6800/MeOH/CHCl₃ | | | | | |
| | wt./wt./wt. | | | | | |
| PCL-2 | 11.1/26.0/62.9 | 48 | 240 | 2.0 | ND | ND |
| | Capa6800/MeOH/CHCl₃ | | | | | |
| | wt./wt./wt. | | | | | |
| PCL-3 | 11.1/26.0/61.9/1 | 48 | 7 | 1.0 | ND | ND |
| | Capa6800/MeOH/ | | | | | |
| | CHCl₃/Sn(II)Oct₂ | | | | | |
| | wt./wt./wt./wt. | | | | | |
| PLA-19 | 11.1/88.9 | 48 | 75 | 2.3 | ND | ND |
| | PL49/MeOH | | | | | |
| | wt./wt. | | | | | |
| PLA-20 | 11.1/26.0/62.9 | 48 | 75 | 3.6 | ND | ND |
| | PL49/water/CHCl₃ | | | | | |
| | wt./wt./wt. | | | | | |
| PLA-21 | 11.1/26.0/62.9 | 48 | 75 | 3.4 | ND | ND |
| | PL49/EtOH/CHCl₃ | | | | | |
| | wt./wt./wt. | | | | | |
| PLA-22 | 11/5/84 | 48 | 75 | 2.1 | ND | ND |
| | PL49/MeOH/CHCl₃ | | | | | |
| | wt./wt./wt. | | | | | |
| PLA-23 | 11/49/40 | 48 | 75 | 2.1 | ND | ND |
| | PL49/MeOH/CHCl₃ | | | | | |
| | wt./wt./wt. | | | | | |
| PLA-24 | 11/46/34 | 48 | 75 | 2.5 | ND | ND |
| | PL49/MeOH/CHCl₃ | | | | | |
| | wt./wt./wt. | | | | | |

*ND = not determined

Examples PLA-7 and 12 consider the effect of reactor volume and reaction time and demonstrate the deleterious effect of greater reactor headspace on the depolymerization reaction. Example PLA-13 considers the effect of inclusion of a catalyst (SnOct₂) in the depolymerization reaction, and demonstrates the greatly enhanced reaction rates resulting therefrom. Example PLA-14 considers the effect of a lower IV of the PLA subjected to depolymerization. PLA-14a is a control to show the base mean IV value of this resin. PLA-14b was subjected to comparable reaction parameters as PLA-11, and demonstrates a reduced reaction rate. Examples PLA-15 and PLA-16 consider the effect of THF as the solvent and MeOH and water, respectively, as the alcohol. These experiments demonstrate that depolymerization reactions can proceed via methanolysis and hydrolysis reactions, respectively, in THF. Example PLA-17 is a further example using THF as solvent and water as alcohol and demonstrates hydrolysis kinetics relative to PLA-16. Example PLA-18 considers the effect of mixing two different PLA resins (PLA-10 and PLA 14a). PLA-18 demonstrates alcoholysis of a 50/50 (wt./wt.) mixture of these resins in the chloroform/methanol system of Example PLA-11.

Examples PET-1 consider the effect of the depolymerization reaction conditions on polyethylene terephthalate, denoted as PET, when contacting the mixture of Example PLA-11. Example PET-1a is a control, providing the molecular weight and PDI for PET bottle flakes and Example PET-1b provides the molecular weight and PDI for PET bottle flakes subjected to reaction in a PLA/CHCl₃/MeOH mixture. The PET in example PET-1b is not substantially dissolved. This example demonstrates that the depolymerization process does not affect PET, e.g., which may be present in co-mingled waste with polyesters such as PLA.

PCL samples were also evaluated. Example PCL-Control provides IV data for (untreated) PCL resin pellets. Example PCL-1 provides IV data for a solution of the PCL resin in a MeOH/CHCl₃ mixture subjected to comparable conditions as PLA-11. Example PCL-2 is comparable to Example PCL-1 with the exception that the reaction time was longer. These examples demonstrate that PCL generally undergoes methanolysis more slowly than PLA (as would be expected, given that PCL has a higher molecular weight between ester linkages than PLA). Example PCL-3 considers the effect of inclusion of a catalyst (SnOct₂) in the depolymerization reaction of PCL, and demonstrates that the methanolysis of PCL proceeds more quickly/effectively in the presence of a catalyst (although still slower than methanolysis of PLA under comparable conditions).

Examples PLA-19 and 20 consider reaction conditions where the PLA is not “substantially dissolved.” PLA-19 considers the effect of excluding the “solvent,” i.e., placing the PLA resin in methanol alone. A precipitate was clearly visible, and reaction would be expected to occur at the resin interface only. This example demonstrates much slower reaction kinetics relative to Example PLA-11. Example PLA-20 considers the effect of incorporating water as the alcohol, resulting in a mixture wherein the interface was visible. This example demonstrates much slower reaction kinetics relative to Example PLA-11. In Example PLA-21 (comparable to Example PLA-20, but with ethanol in place of the water), no interface was visible, and this example demonstrates slower reaction kinetics relative to PLA-11 (likely due to the bulkier alcohol). Example PLA-22 (1:2.2 alcohol:PLA) maintains substantial dissolution of the polymer (with no interface visible), but exhibits slower reaction kinetics because of the reduced alcohol:PLA ratio relative to Example PLA-11. Examples PLA-23 and PLA-24 had alcohol:PLA ratios of 4.5:1 and 2.3:1, respectively, and both exhibited visible precipitates. These examples also demonstrate slower reaction kinetics relative to Example PLA-11.

1. A method for depolymerizing a polymer, comprising:
obtaining the polymer, wherein the polymer comprises hydrolyzable linkages; and
obtaining a polymeric mixture by at least contacting the polymer with a solvent and an alcohol,
wherein the polymer in the polymeric mixture is substantially dissolved, and
wherein the contacting is conducted at a temperature at or below a boiling point of the polymeric mixture,
to provide a depolymerized material.

2. The method of claim 1, wherein the polymeric mixture exhibits no visible phase separation to the naked eye.

3. The method of claim 1, wherein the polymer comprises, at least in part, a polyester.

4. The method of claim 1, wherein the polymer is a copolymer.

5. The method of claim 1, wherein the polymer comprises a polymer selected from a group consisting of polysaccharides; chitin; chitosan; proteins; polyglycolides; poly(caprolactones); poly(hydroxybutyrates); poly(anhydrides); aliphatic polycarbonates; polyesters, poly(orthoesters); poly(amino acids); poly(ethylene oxide); polyphosphazenes, polyvinyl alcohols, copolymers thereof, and derivatives thereof.

6. The method of claim 1, wherein the hydrolyzable linkages comprise ester linkages.

7. The method of claim 1, wherein the polymer is poly(lactic acid), a derivative of poly(lactic acid), or a copolymer of poly(lactic acid).

8. The method of claim 1, wherein the solvent is selected from a group consisting of methylene chloride, chloroform, tetrachloromethane, trichloroethylene, tetrahydrofuran, acetone, ethyl acetate, dioxane, N-methyl pyrrolidone, hexafluoroisopropanol, 2,2,2-trifluoroethanol, acetophenone, acetonitrile, toluene, cyclohexanone, butylbenzoate, isophorone, nitropropane, methylethyl ketone, dimethylacetamide, benzylbenzoate, caprolactone, tributyl phosphate, benzene, dimethylformamide, and any combinations thereof.

9. The method of claim 1, wherein the polymeric mixture is a solution, and wherein the polymer is completely dissolved.

10. The method of claim 1, wherein the polymeric mixture is a gel.

11-30. (canceled)

31. A system, comprising:
one or more mixing modules configured to obtain a polymer comprising hydrolyzable linkages and to obtain a polymeric mixture by at least contacting the polymer with a solvent and an alcohol; and
one or more reactors configured to provide a depolymerized material based at least in part by conducting contacting between the polymer, the solvent, and the alcohol at a temperature at or below a boiling point of the polymeric mixture such that the polymer is substantially dissolved.

32. The system of claim 31, wherein at least one of the one or more mixing modules obtain the polymer based at least in part on receiving the polymer from an input source.

33. The system of claim 31, wherein at least one of the one or more mixing modules obtain the polymer based at least in part on retrieving the polymer from an input source.

34. The system of claim 31, wherein at least one of the one or more reactors comprises at least one of the one or more mixing modules.

35. The system of claim 31, wherein at least one of the one or more reactors is a continuous stirred tank reactor, a batch reactor, or a plug flow reactor.

36. The system of claim 31, further comprising: one or more heating modules configured to provide heat to the polymeric mixture.

37. The system of claim 36, wherein at least one of the one or more reactors comprises, or is operatively connected to, at least one of the one or more heating modules.

38. The system of claim 31, further comprising one or more separating modules configured to separate the depolymerized material.

39-47. (canceled)

48. Depolymerized material obtained by contacting a polymer comprising hydrolyzable linkages, a solvent, and an alcohol, wherein the contacting is conducted at a temperature at or below a boiling point of a polymeric mixture comprising the polymer, the solvent, and the alcohol, and wherein the polymer in the polymeric mixture is substantially dissolved.

49. The depolymerized material of claim 48, wherein the depolymerized material is one or more of lactic acid, methyl lactate, poly(lactic acid) oligomers, and lactate oligomer.
